(19) United States
(12) Patent Application Publication
(10) Pub. No.: US 2022/0331140 A1
(43) Pub. Date: Oct. 20, 2022

(54) Shoulder Brace

(71) Applicant: Roessingh Revalidatie Techniek B.V., Enschede (NL)

(72) Inventors: John-John de Koning, Enschede (NL); Reinout Otto van Vliet, Lonneker (NL)

(21) Appl. No.: 17/722,868

(22) Filed: Apr. 18, 2022

(30) Foreign Application Priority Data
Apr. 19, 2021 (EP) 21169085.4

(51) Int. Cl. A61F 5/37

(52) U.S. Cl. CPC A61F 5/3723

(57) ABSTRACT

The invention relates to a shoulder brace for approximation of the humerus head into the shoulder joint of a person, which the shoulder brace includes a shoulder pad adapted to be positioned over the top of a shoulder of a person which he shoulder pad has a dorsal end and a ventral end, and which the shoulder pad configured to extend from the dorsal side over the top of the shoulder to the ventral side; and an adjustable armpit belt. Both ends of the adjustable armpit belt are arranged to the shoulder pad, which the adjustable armpit belt is adapted to run along the armpit opposite of the shoulder on top of which the shoulder pad is positioned; an upper arm sleeve adapted to be worn around the upper arm of a person and configured to enclose the upper arm and extend from the ventral side to the dorsal side of the upper arm; and coupling means arranged with one end to the shoulder pad and with the other end to the upper arm sleeve. The coupling means include at least one flexible tube running over the shoulder pad from the dorsal end to the ventral end and at least one wire running through the at least one flexible tube. Both ends of the at least one wire are arranged to the upper arm sleeve on opposite sides relative to the axis of the upper arm sleeve.

### CROSS-REFERENCE TO RELATED APPLICATION

This application claims priority to European Patent Application No. 21169085.4 filed Apr. 19, 2021, the disclosure of which is hereby incorporated by reference in its entirety.

### BACKGROUND OF THE INVENTION

### Field of the Invention

The invention relates to a shoulder brace for approximation of the humerus head into the shoulder joint of a person, which shoulder brace comprises:
a shoulder pad adapted to be positioned over the top of a shoulder of a person, which shoulder pad has a dorsal end and a ventral end, and which shoulder pad configured to extend from the dorsal side over the top of the shoulder to the ventral side;
an adjustable armpit belt, wherein both ends of the adjustable armpit belt are arranged to the shoulder pad, which adjustable armpit belt is adapted to run along the armpit opposite of the shoulder on top of which the shoulder pad is positioned;
an upper arm sleeve adapted to be worn around the upper arm of a person and configured to enclose the upper arm and extend from the ventral side to the dorsal side of the upper arm; and
coupling means arranged with one end to the shoulder pad and with the other end to the upper arm sleeve.

### Description of Related Art

Such a shoulder brace is known from U.S. Pat. Nos. 5,403,268 and 3,235,474.

Good positioning of the humerus head according to the joint surface will prevent or reduce the anterior and/or caudal (sub)luxation of humerus head.

This subluxation in most of the cases due to dysbalance of shoulder stabilizing muscles as a result of central neurological causes (like stroke), plexopathies, neuromuscular disorders or serious hypermobility syndromes (like Ehlers Danlos Syndrome). Traction of the shoulder structures caused by the weight and movements of the arm causes pain by local elongation of the joint capsule, microdamage of the glenohumeral joint or overload of residual stabilizing rotator cuff muscles because of overcompensation

A commonly known manner of relieving the shoulder is by use of a sling. The sling is wrapped around the forearm and around the neck of the person. As a result the weight of the arm is borne by the neck and the arm is immobilized against the body. The neck is however not suited for carrying such a weight and after a day wearing a sling, the muscles in the neck will start to ache. Also, the fact that one of the arms cannot be properly used, as it is immobilized, will result in overloading other parts of the body, such as the other arm and the back.

Improved slings are known in the prior art, in which the sling does not run over the neck, but runs over the shoulder. This relieves the neck, but the forearm is still immobilized by the sling being wrapped around it.

Another shoulder brace is known from U.S. Pat. Nos. 5,403,268 and 3,23,5474. Such a shoulder brace has a shoulder pad held in position by an adjustable arm pit belt. An upper arm sleeve is furthermore provided and mounted by one or more straps to the shoulder pad.

Such a shoulder brace allows for more freedom of movement compared to for example a sling. The shoulder brace provides support when the upper arm is hanging downwards, but as soon as the upper arm is lifted, the distance between the mounting points on the upper arm sleeve and the shoulder pad changes, such that the support for the shoulder is changed.

So, the known shoulder braces either provide insufficient support, such that the shoulder is not relieved sufficiently and might cause blood circulation problems, or the known shoulder braces provide sufficient support, while immobilizing the full arm.

### SUMMARY OF THE INVENTION

It is an object of the invention to reduce or even remove the above mentioned disadvantages.

This object is achieved according to the invention with a shoulder brace according to the preamble, which is characterized in that the coupling means comprise:
at least one flexible tube running over the shoulder pad from the dorsal end to the ventral end;
at least one wire running through the at least one flexible tube, wherein both ends of the at least one wire are arranged to the upper arm sleeve on opposite sides relative to the axis of the upper arm sleeve.

When the shoulder brace is worn by a user, the at least one flexible tube thus extends in a plane substantially parallel to the sagittal plane.

The at least one wire is able to slide through the at least one flexible tube. So, when the person wearing the shoulder brace moves the upper arm back and forth parallel to the sagittal plane, the increased distance between a mounting point on one side on the upper arm sleeve and the shoulder pad is compensated, as the wire can slide though the flexible tube, by the decreased distance of a mounting on the opposite side of the upper arm sleeve.

In a preferred embodiment of the shoulder brace according to the invention at least one section of the at least one flexible tube is fixed to the shoulder pad and wherein both ends of the at least one flexible tube are arranged free.

If the upper arm is moved in the frontal plane, the distance between the shoulder pad and the mounting of the end of the wire will increase. By providing the flexible tube with free ends, these ends can bend towards the movement of the upper arm such that the distance between the shoulder pad and the mounting of the ends on the upper arm sleeve are maintained more or less equal.

In a preferred embodiment of the shoulder brace according to the invention the free ends of the at least one flexible tube are at least 5 cm long. This allows for sufficient flexibility to allow for movement of the upper arm in the frontal plane.

In yet a further preferred embodiment of the shoulder brace according to the invention at least two flexible tubes run parallel over the shoulder pad from the dorsal end to the ventral end and wherein wires run through each of the at least two flexible tubes and wherein both ends of each wire are arranged to the upper arm sleeve on opposite sides relative to the axis of the upper arm sleeve.

Having at least two flexible tubes with wires running there through provides a more stable support of the upper arm sleeve and thus for a more stable support of the shoulder.

In an even more preferred embodiment of the shoulder brace according to the invention the ends of both wires on one side of the upper arm sleeve are connected to each other and guided around a guide mounted on said one side of the upper arm sleeve.

By connecting the ends of both wires on one side of the upper arm sleeve, a single wire is formed, which runs through a first tube along the guide and back through the second tube. This further improves the stable support of the shoulder.

In another embodiment of the shoulder brace according to the invention the flexibility of the free ends of the two flexible tubes differ between the two flexible tubes. A more stiff tube will compensate less the distance change when the upper arm is moved in the frontal plane.

In a further preferred embodiment of the shoulder brace according to the invention at least one end of the at least one wire is linked to a lockable spool rotationally arranged on the upper arm sleeve.

The spool allows for a quick adjustment of the length of the wires, such that the shoulder brace can be fitted on a specific person.

Preferably, the shoulder brace further comprises a tightening mechanism attached to the upper arm sleeve and coupled to the spool, the tightening mechanism having a spring for winding a first length of cable around the spool and a manual control for manually winding a second length of cable around the spool to adjust the length of the wire.

The spring allows for any play on the wire, which contributes to the wear comfort of the shoulder brace according to the invention.

### BRIEF DESCRIPTION OF THE DRAWINGS

These and other features of the invention will be elucidated in conjunction with the accompanying drawings.

FIG. 1 shows a front view of a person wearing an embodiment of a shoulder brace according to the invention.

FIG. 2 shows a back view of the person of FIG. 1.

FIG. 3 shows a detailed view of the person of FIG. 1 with his upper arm lifted in the frontal plane.

### DESCRIPTION OF THE INVENTION

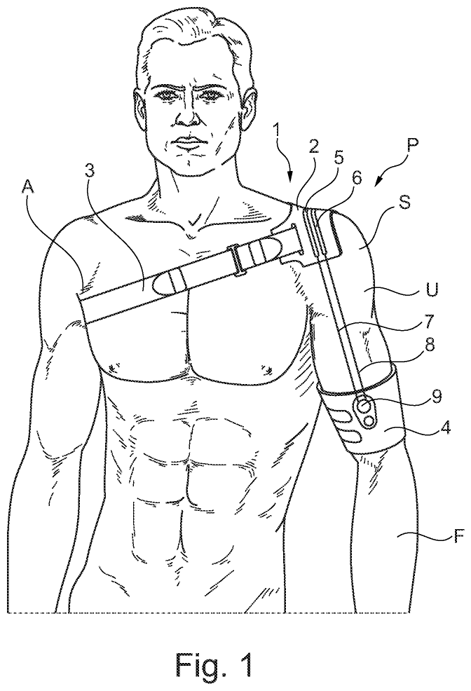

FIG. 1 shows a person P wearing a shoulder brace 1 according to the invention. The shoulder brace 1 has a shoulder pad 2 arranged over the shoulder S of the person P. An adjustable arm pit belt 3, which is attached to the shoulder pad 2 and runs under the arm pit A, keeps the shoulder pad 2 in position. Furthermore, an upper arm sleeve 4 is arranged around the upper arm U near the elbow where the forearm F starts.

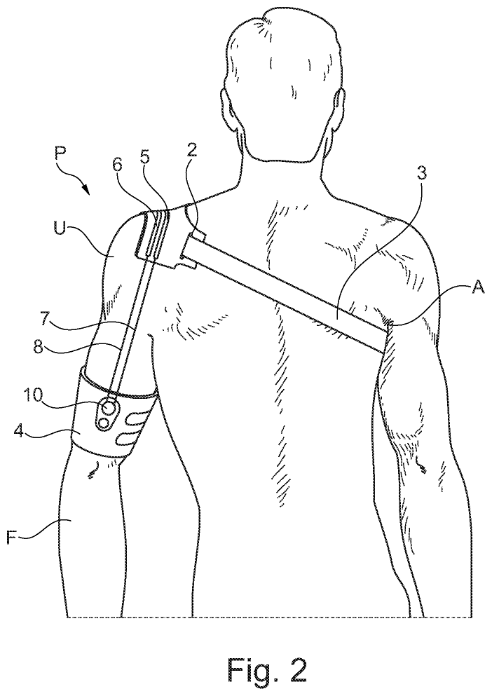

The shoulder pad 2 is provided with two flexible tubes 5, 6 which run over the shoulder pad 2 from the dorsal end (see FIG. 2) to the ventral end (see FIG. 1). Two wires 7, 8 run through the flexible tubes 5, 6 respectively and are wound with one end on a spool 9 and are connected with the other ends to each other and are guided along a guide 10 (see FIG. 2).

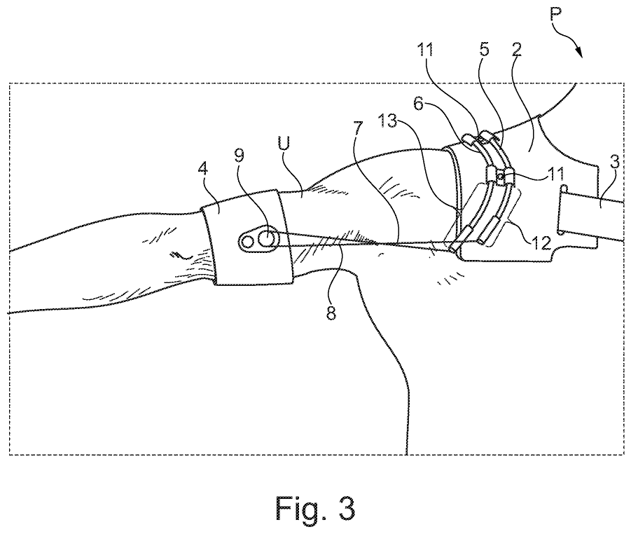

FIG. 3 shows a more detailed view of the person P of FIG. 1 with his upper arm U lifted up in the frontal plane.

The flexible tubes 5, 6 are mounted to the shoulder pad 2 with mounts 11, leaving each flexible tube 5, 6 with a free end 12, 13 respectively.

As shown, the free ends 12, 13 of the flexible tubes 5, 6 will bend when the person P lifts his upper arm U in the frontal plane and compensate for the change in distance between the spool 9 and the ends of the flexible tubes 5, 6 if they were fixed to the shoulder pad 2 and would not have been free.

1. A shoulder brace for approximation of the humerus head into the shoulder joint of a person, which shoulder brace comprises:
a shoulder pad adapted to be positioned over the top of a shoulder of a person, which shoulder pad has a dorsal end and a ventral end, and which shoulder pad configured to extend from the dorsal side over the top of the shoulder to the ventral side;
an adjustable armpit belt, wherein both ends of the adjustable armpit belt are arranged to the shoulder pad, which adjustable armpit belt is adapted to run along the armpit opposite of the shoulder on top of which the shoulder pad is positioned;
an upper arm sleeve adapted to be worn around the upper arm of a person and configured to enclose the upper arm and extend from the ventral side to the dorsal side of the upper arm; and
coupling means arranged with one end to the shoulder pad and with the other end to the upper arm sleeve,
wherein the coupling means comprise:
at least one flexible tube running over the shoulder pad from the dorsal end to the ventral end; and
at least one wire running through the at least one flexible tube, wherein both ends of the at least one wire are arranged to the upper arm sleeve on opposite sides relative to the axis of the upper arm sleeve.

2. The shoulder brace according to claim 1, wherein at least one section of the at least one flexible tube is fixed to the shoulder pad and wherein both ends of the at least one flexible tube are arranged free.

3. The shoulder brace according to claim 2, wherein the free ends of the at least one flexible tube are at least 5 cm long.

4. The shoulder brace according to claim 1, wherein at least two flexible tubes run parallel over the shoulder pad from the dorsal end to the ventral end and wherein wires run through each of the at least two flexible tubes and wherein both ends of each wire are arranged to the upper arm sleeve on opposite sides relative to the axis of the upper arm sleeve.

5. The shoulder brace according to claim 4, wherein the ends of both wires on one side of the upper arm sleeve are connected to each other and guided around a guide mounted on said one side of the upper arm sleeve.

6. The shoulder brace according to claim 4, wherein the flexibility of the free ends of the two flexible tubes differ between the two flexible tubes.

7. The shoulder brace according to claim 1, wherein at least one end of the at least one wire is linked to a lockable spool rotationally arranged on the upper arm sleeve.

8. The shoulder brace according to claim 7, further comprising a tightening mechanism attached to the upper arm sleeve and coupled to the spool, the tightening mechanism having a spring for winding a first length of cable around the spool and a manual control for manually winding a second length of cable around the spool to adjust the length of the wire.
